(19) United States
(12) Patent Application Publication
(10) Pub. No.: US 2026/0174497 A1
(43) Pub. Date: Jun. 25, 2026

(54) ULTRASOUND TRANSDUCER CATHETER

(71) Applicant: Medtronic Ireland Manufacturing Unlimited Company, Dublin (IE)

(72) Inventors: Gerry Oliver McCaffrey, Abbeyknockmoy (IE); Paul J. Coates, Corte Madera, CA (US)

(21) Appl. No.: 18/866,145

(22) PCT Filed: May 22, 2023
(86) PCT No.: PCT/EP2023/063574

§ 371 Date: Nov. 15, 2024

Related U.S. Application Data
(60) Provisional application No. 63/365,449, filed on May 27, 2022.

(51) Int. Cl. A61B 18/14; A61B 8/00; A61M 25/00

(52) U.S. Cl. CPC A61B 18/1492; A61B 8/445; A61M 25/0068; A61M 25/0074

(57) ABSTRACT

A catheter including an expandable member, an outer catheter body including an outer catheter lumen, an inner tube positioned within the outer catheter lumen and defining an inner tube lumen, and an ultrasound transducer positioned in the expandable member. The transducer may include a transducer body comprising an inner surface defining a transducer lumen, wherein the transducer lumen is fluidically connected to the inner tube lumen, a transducer support member positioned at a distal portion of the transducer body and defining one or more fluid apertures through which the fluid is configured to flow from the transducer lumen into the expandable member, and a plurality of electrodes extending along at least a first portion of an outer surface of and at least a second portion of the inner surface of the transducer body.

### TECHNICAL FIELD

The present technology is related to catheters.

### BACKGROUND

Catheters including one or more energy delivery elements have been proposed for use in various medical procedures, including neuromodulation procedures. For example, some catheters include an ultrasound transducer configured to deliver ultrasound energy to a region of tissue during an ablation procedure.

### SUMMARY

The present disclosure describes catheters that include an ultrasound transducer positioned within an expandable member, where the catheter is configured to deliver a cooling medium to an interior volume of the expandable member to cool the ultrasound transducer. In some examples, the catheter is configured to deliver the cooling medium to cool an inside surface of the ultrasound transducer. For example, the ultrasound transducer can include a transducer body including an inner surface defining a transducer lumen, and a proximal portion of the transducer body may be connected to a distal portion of an inner tube defining an inner tube lumen. The inner tube lumen is fluidically connected to the transducer lumen and is configured to deliver the cooling medium into the transducer lumen to cool the inner surface of the transducer body.

In some examples, the catheter is configured to reduce transmission of ultrasound energy across the transducer lumen to help reduce an operating temperature of the ultrasound transducer. For example, the catheter may include a second expandable member and the ultrasound transducer may be disposed on an outer surface of the second expandable member. The second expandable member may be filled with a medium (e.g., a gas) that is configured to impede the transmission of ultrasound energy.

The configuration of the catheters described in this disclosure may improve the cooling of the ultrasound transducer by at least reducing the transmission of heat to other areas of the respective catheter and/or increase the cooling rate of the ultrasound transducer.

In some examples, the catheters described herein may be useful for neuromodulation within a blood vessel or a body lumen other than a vessel, for extravascular neuromodulation, for non-renal-nerve neuromodulation, and/or for use in therapies other than neuromodulation.

In some examples, the disclosure describes a catheter including an expandable member; an outer catheter body comprising an outer catheter lumen in fluid communication with the expandable member; an inner tube positioned within the outer catheter lumen, the inner tube defining an inner tube lumen configured to deliver a fluid to the expandable member; and an ultrasound transducer positioned in the expandable member, the transducer comprising: a transducer body comprising an inner surface defining a transducer lumen, wherein at least a proximal portion of the transducer body is connected to a distal portion of the inner tube to fluidically connect the inner tube lumen and the transducer lumen; a transducer support member positioned at a distal portion of the transducer body, the transducer support member defining one or more fluid apertures through which the fluid is configured to flow from the transducer lumen into the expandable member; and a plurality of electrodes extending along at least a first portion of an outer surface of the transducer body and at least a second portion of the inner surface of the transducer body.

In some examples, the disclosure describes a catheter comprising: a first expandable member; a second expandable member disposed within the first expandable member; an outer catheter body defining an outer catheter lumen in fluid communication with the first expandable member; a fluid delivery tube disposed within the outer catheter lumen, the fluid delivery tube configured to deliver a first medium to the first expandable member; an inner tube disposed within the outer catheter lumen, the inner tube defining an inner tube lumen configured to deliver a second medium to the second expandable member to expand the second expandable member; and an ultrasound transducer disposed on an outer surface of the second expandable member.

In some examples, the disclosure describes A method comprising: navigating a catheter through vasculature of a patient to a target treatment site; expanding an expandable member of the catheter and circulating a fluid through the expandable member, wherein expanding the expandable member and circulating the fluid comprises: introducing the fluid into the expandable member through an inner tube lumen defined by an inner tube of the catheter, wherein the catheter further comprises an outer catheter body defining an outer catheter lumen in fluid communication with the expandable member, and wherein the inner tube is disposed within the outer catheter lumen; introducing the fluid into a transducer lumen of an ultrasound transducer of the catheter, the ultrasound transducer being positioned radially inward of the expandable member, wherein the ultrasound transducer comprises a transducer body comprising an inner surface defining the transducer lumen, and wherein at least a proximal portion of the transducer body is connected to a distal portion of the inner tube to fluidically connect the inner tube lumen and the transducer lumen, wherein the fluid flows from the transducer lumen into the expandable member through one or more fluid apertures of a transducer support lumen positioned at a distal portion of the transducer body, and from the expandable member to the outer catheter lumen; and delivering therapy to the target treatment site via a plurality of electrodes extending along at least a first portion of an outer surface of the transducer body and at least a second portion of the inner surface of the transducer body.

In some examples, the disclosure describes A method comprising: navigating a catheter through vasculature of a patient to a target treatment site, the catheter comprising: a first expandable member; a second expandable member disposed within the first expandable member; an outer catheter body defining an outer catheter lumen in fluid communication with the first expandable member; a fluid delivery tube disposed within the outer catheter lumen, the fluid delivery tube configured to deliver a first fluid to the first expandable member; an inner tube disposed within the outer catheter lumen, the inner tube defining an inner tube lumen configured to deliver a second fluid to the second expandable member; and an ultrasound transducer disposed on an outer surface of the second expandable member; expanding the first expandable member of the catheter, wherein expanding the first expandable member comprises introducing a first medium into the first expandable member through the fluid delivery tube; expanding the second expandable member of the catheter, wherein expanding the second expandable member comprises introducing a second medium into the second expandable member through the inner tube; circulating the first medium through the first expandable member using the fluid delivery tube and the outer catheter lumen; and delivering a therapy to the target treatment site via the ultrasound transducer.

Further disclosed herein is a catheter including an expandable member, an outer catheter body including an outer catheter lumen, an inner tube positioned within the outer catheter lumen and defining an inner tube lumen, and an ultrasound transducer positioned in the expandable member, wherein the transducer may include a transducer body comprising an inner surface defining a transducer lumen, wherein the transducer lumen is fluidically connected to the inner tube lumen, a transducer support member positioned at a distal portion of the transducer body and defining one or more fluid apertures through which the fluid is configured to flow from the transducer lumen into the expandable member, and a plurality of electrodes extending along at least a first portion of an outer surface of and at least a second portion of the inner surface of the transducer body.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of an example catheter that includes an expandable member and an ultrasound transducer.

FIG. 2 is a conceptual diagram illustrating a cross-sectional view of an example distal portion of the catheter of FIG. 1, the cross-section being taken along line A-A in FIG. 1.

FIG. 3 is a conceptual diagram illustrating a cross-sectional view of the example distal portion of the catheter of FIG. 2, the cross-section being taken along line B-B in FIG. 2.

FIG. 4 is a conceptual diagram illustrating a cross-sectional view of the example distal portion of the catheter of FIG. 2, the cross-section being taken along line C-C in FIG. 2.

FIG. 5 is a conceptual diagram illustrating a cross-sectional view of another example distal portion of the catheter of FIG. 1, the cross-section being taken along line A-A in FIG. 1.

FIG. 6 is a conceptual diagram illustrating a cross-sectional view of the example distal portion of the catheter of FIG. 5, the cross-section being taken along line D-D in FIG. 5.

FIG. 7 is a conceptual diagram illustrating a cross-sectional view of the example distal portion of the catheter of FIG. 5, the cross-section being taken along line E-E in FIG. 5.

FIG. 8 is a conceptual diagram illustrating a cross-sectional view of another example distal portion of the catheter of FIG. 1, the cross-section being taken along line A-A in FIG. 1.

FIG. 9 is a flow diagram illustrating an example process of delivering ultrasound therapy to a patient using a catheter that includes an expandable member.

FIG. 10 is a flow diagram illustrating another example process of delivering ultrasound therapy to a patient using another catheter that includes an expandable member.

FIG. 11 is a conceptual illustration of an example process for accessing a renal artery and modulating renal nerves with the catheter of FIG. 1.

FIG. 12 is a conceptual illustration of an example sympathetic nervous system (SNS) illustrating how the brain communicates with the body via the SNS.

FIG. 13 is an enlarged anatomic view of nerves innervating a left kidney to form the renal plexus surrounding the left renal artery.

FIG. 14 is an anatomic view of a human body depicting neural efferent and afferent communication between the brain and kidneys.

FIG. 15 is a conceptual view of a human body depicting neural efferent and afferent communication between the brain and kidneys.

FIG. 16 is an anatomic view of the arterial vasculature of a human.

FIG. 17 is an anatomic view of the venous vasculature of a human.

### DETAILED DESCRIPTION

In examples described herein, a catheter includes an ultrasound transducer positioned within an expandable member and is configured to deliver ultrasound energy. The ultrasound transducer includes a transducer body and two or more electrodes. In some examples, the ultrasound transducer is configured to generate the ultrasound energy by at least converting an electric current, e.g., an alternating current (AC), into sound waves, e.g., ultrasound. For example, the ultrasound transducer may apply an AC electrical signal across a piezoceramic material of the ultrasound transducer body via the electrodes, which causes the piezoceramic material to vibrate at a given frequency to generate the ultrasound energy. As a result of the vibrations, the ultrasound transducer may generate heat. The expandable member of catheters described herein is configured to be filled with a medium to aid the transmission of the ultrasound energy to the tissue of the patient and to aid in the cooling of the ultrasound transducer.

Some ultrasound transducers are configured to transmit ultrasound energy in multiple directions, e.g., radially inwards and radially outwards. When the ultrasound transducer transmits the ultrasound energy radially inwards, the ultrasound energy may be transmitted to the one or more parts of the catheter, which may lead to the buildup of heat in the one or more parts of the catheter (e.g., in the ultrasound transducer). For example, in some catheter configurations, the ultrasound transducer is supported within the expandable member by one or more support members, and ultrasound energy transmitted towards the support members may inadvertently heat the support members. The heat buildup in the one or more support members may lead to undesirable heating of the catheter or particular parts of the catheter during operation of the ultrasound transducer.

The example catheters described in this disclosure are configured to improve the cooling of the ultrasound transducer. The example catheters may improve the cooling of the ultrasound transducer by at least reducing heat buildup in the catheter, increasing heat dissipation from the ultrasound transducer into a cooling medium, or the like or combinations thereof. In some examples, a catheter is configured to deliver the cooling medium to cool an inside surface of the ultrasound transducer. For example, an inner surface of the transducer body can define a transducer lumen that extends through the transducer body. The transducer lumen is fluidically connected to one or more cooling fluid delivery tubes (e.g., an inner tube defining an inner tube lumen) disposed within an elongated body of the catheter. The transducer lumen may facilitate the flow of the cooling medium across the inner surface of the transducer body, which may increase the transfer of heat out of the ultrasound transducer and into the cooling medium.

In some examples, the ultrasound transducer is supported by one or more transducer support members at a distal portion of the ultrasound transducer. Portions of the ultrasound transducer proximal to the one or more transducer support members may be unsupported, which provides more space radially inward of the ultrasound transducer for a cooling fluid to flow within the inner lumen of the ultrasound transducer to cool the ultrasound transducer and facilitate the transfer of heat out of the ultrasound transducer. In addition, heat buildup in the one or more transducer support members may be reduced by reducing the size of the transducer support members, e.g., relative to a configuration in which the support members extend along more of the interior surface of the ultrasound transducer.

In some examples, the catheter is configured to reduce transmission of ultrasound energy across the transducer lumen to help reduce an operating temperature of the ultrasound transducer. For example, in some examples, the catheter includes a second expandable member disposed within the expandable member (also referred to herein as an outer expandable member or a first expandable member) and the ultrasound transducer is disposed radially outwards of the second expandable member, e.g., on an outer surface of the second expandable member. The second expandable member may be filled with a second medium (e.g., a gas) that impedes the transmission of the ultrasound energy radially inwards of the catheter and reduces overall heat buildup in the catheter. In some examples, the second medium is different from the cooling medium.

Although neuromodulation is primarily referred to herein, the catheters described herein may be used for medical procedures other than neuromodulation, including electrical stimulation therapy.

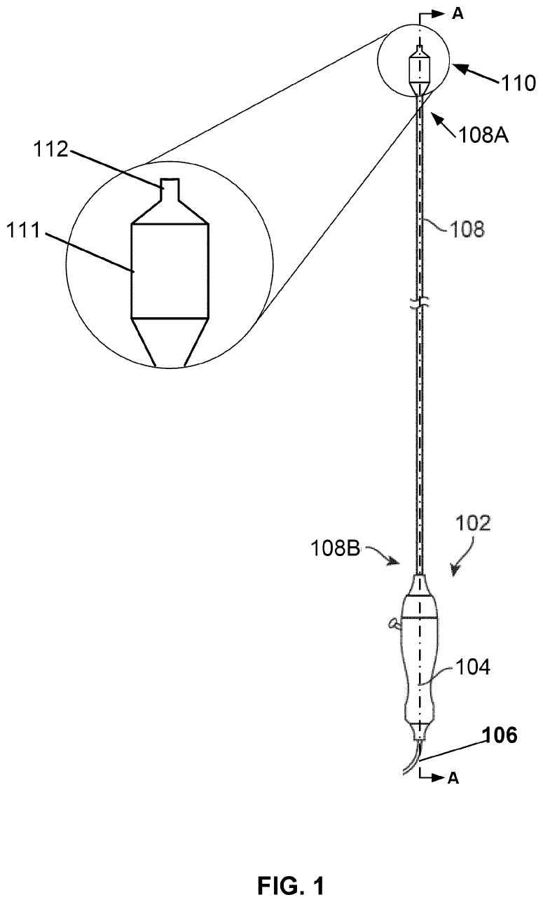

FIG. 1 is a partial schematic illustration of an example catheter 102 that includes an expandable member 111 and an ultrasound transducer (described in further detail below). Catheter 102 includes a handle 104, an elongated body 108 attached to handle 104, and at least one therapeutic element 110 carried by elongated body 108. Therapeutic element 110 includes expandable member 111 and an ultrasound transducer (not pictured) disposed within expandable member 111. Elongated body 108 includes a distal portion 108A and a proximal portion 108B. Distal portion 108A includes a distal end 112 of catheter 102 and therapeutic element 110. In some examples, therapeutic element 110 may be positioned proximal to distal end 112.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device. For example, distal portion 108A of elongated body 108 refers to a portion of elongated body 108 at a position distant from the clinician and proximal portion 108B or elongated body 108 refers to a portion of elongated body 108 at a position near the clinician. In some examples, distal portion 108A is a distalmost portion of catheter 102 including a distal end of catheter 102.

Elongated body 108 may have any suitable outer diameter, and the diameter can be constant along the length of elongated body 108 or may vary along the length of elongated body 108. In some examples, elongated body 108 can be 2, 3, 4, 5, 6, or 7 French or another suitable size. An outer layer of elongated body 108 may be formed from any suitable material or combination of materials, such as, but not limited to, one or more of nylon or a thermoplastic such as polyethylene terephthalate (PET), parylene, polyvinyl chloride (PVC), polyethylene, ethylene chlorotrifluoroethylene (ECTFE), or polyvinylidene fluoride (PVDF).

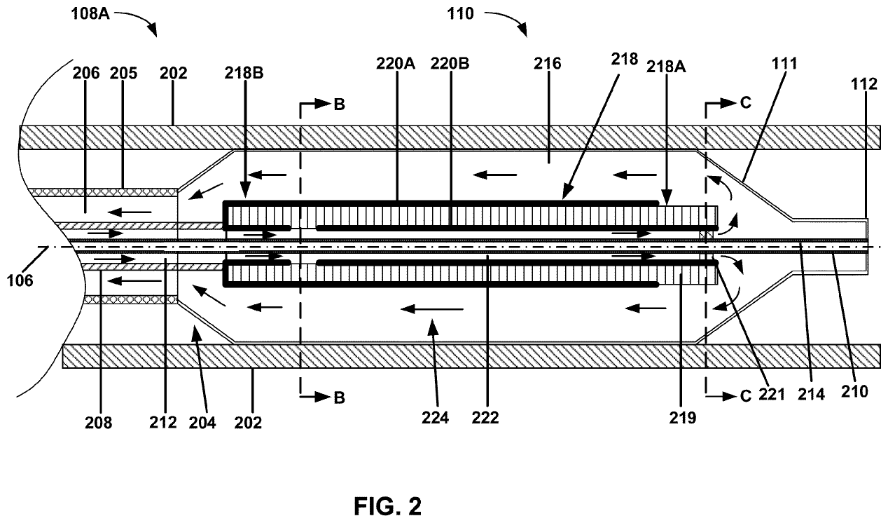

Therapeutic element 110 includes an ultrasound transducer (shown in FIG. 2) configured to deliver ultrasound energy at a target tissue site (also referred to herein as “target region”) within a patient when distal portion 108A is positioned proximate the target tissue site. While ultrasound energy is primarily referred to herein, in other examples, therapeutic element 110 can be configured to deliver other types of therapeutic energies (e.g., microwave energy). The ultrasound transducer may deliver the ultrasound energy in response to input (e.g., an electrical current) from a medical device (also referred to herein as a “therapy delivery device”). Medical device may include and/or may be electrically connected to a power source. The power source may be external to the body of the patient. Although a single ultrasound transducer is referred to herein, in other examples, catheter 102 can include a plurality of ultrasound transducers. In some examples, the plurality of ultrasound transducers may be radially disposed around a longitudinal axis 106 of catheter 102. In some examples, the plurality of ultrasound transducers may be connected, e.g., to form a single transducer body within expandable member 111.

Expandable member 111 is configured to expand radially outwards when an inflation fluid, which can include a cooling medium described herein in some examples, is delivered to an interior volume of expandable member 111. Expandable member 111 can be, for example, a balloon (e.g., a compliant balloon). In some examples, when expanded, expandable member 111 is symmetrical (e.g., radially symmetrical and/or longitudinally symmetrical). In some examples, when expanded, expandable member 111 is asymmetrical longitudinally along longitudinal axis 106 and/or radially around longitudinal axis 106.

As discussed in further detail with reference to FIG. 2, elongated body 108 includes an outer catheter body which defines an outer catheter lumen. The outer catheter lumen may be in fluid communication with an inner volume of expandable member 111. A clinician may introduce an inflation fluid into and/or extract the inflation fluid from the inner volume of expandable member 111 via the outer catheter lumen, e.g., via a port on proximal portion 108B. In some examples, the port may be proximal to or defined by handle 104.

Introduction of the inflation fluid into the inner volume of expandable member 111 expands expandable member 111 from a collapsed configuration to an expanded configuration. The inflation fluid (also referred to as the “fluid”), is biocompatible. In some examples, the inflation fluid also acts as a cooling medium that cools the ultrasound transducer. The fluid may also be configured to facilitate transmission from the ultrasound transducer to the tissue of the patient. In some examples, the fluid may be a liquid such as sterile water or saline. The fluid can be selected to be biocompatible. In addition, in some examples, the fluid includes a contrast media, e.g., mixed with sterile water.

For example, catheter 102 can be configured such that the inflation fluid circulates, e.g., by circulating the fluid around the ultrasound transducer inside the inner volume of expandable member 111 and transfers heat from the ultrasound transducer. The fluid can be removed from the inner volume of expandable member 111 to transition expandable member 111 from the expanded configuration into the collapsed configuration and/or to enable additional fluid to be introduced into the inner volume of expandable member 111 to further cool the ultrasound transducer. This may be referred to as cycling of the fluid. Because the fluid within expandable member 111 may heat-up as a result of the delivery of the ultrasound energy through the fluid and/or via thermal transfer from the ultrasound transducer, it may be desirable to exchange the warmed fluid for cooler fluid during at least part of a medical procedure. This exchange of fluid, which can be referred to as cycling of the fluid (though the same fluid may not be re-used during the medical procedure), may be performed continuously or periodically during the medical procedure.

In some examples, catheter 102 is configured to accommodate a variety of vessel diameters. For example, renal vessels may have a diameter between about 3 mm and about 8 mm. Other vessels may have other diameters. Distal portion 108A may accommodate different vessel diameters by having expandable member 111 expand to different diameters. In this way, a single catheter 102 may be used to deliver therapy to vessels with different diameters, e.g., diameters in a range of between about 2 mm and about 10 mm.

Distal portion 108A of elongated body 108 is configured to be advanced within a hollow anatomical structure (e.g., a blood vessel) of a human patient to locate expandable member 111 at a target region (e.g., a target treatment site) within or otherwise proximate to the hollow anatomical structure. For example, elongated body 108 may be configured to position expandable member 111 within a blood vessel, a ureter, a urethra, a duct, an airway, or another naturally occurring lumen within the human body. The examples described herein focus on the anatomical structure being a blood vessel, such as a renal vessel, but it will be understood that similar techniques may be used with other hollow anatomical structures.

In certain examples, intravascular delivery of catheter 102 includes percutaneously inserting a guidewire (not shown in FIG. 1) into a vessel of a patient and moving elongated body 108 along the guidewire until expandable member 111 reaches a target treatment site (e.g., a renal artery). For example, catheter 102 can include a guidewire tube or another structure that defines a guidewire lumen configured to receive the guidewire for delivery of catheter 102 using over-the-wire (OTW) or rapid exchange (RX) techniques. In other examples, distal portion 108A may include a steerable or non-steerable device configured for use without a guidewire. In still other examples, catheter 102 can be configured for delivery via an inner catheter or sheath (not shown in FIG. 1), or other inner member (e.g., a guide catheter). In some examples, catheter 102 can be configured to accept an inner catheter within the outer catheter lumen and/or the inner tube lumen. In some examples, the inner member may be a navigation wire (e.g., a guidewire or the like). In some examples, the navigation wire may be disposed on an outer surface of elongated body 108 of catheter 102.

Once at the target treatment site, the clinician may transition expandable member 111 from the collapsed configuration into the expanded configuration by introducing the fluid into the inner volume of expandable member 111, e.g., via the outer catheter lumen or via an inner tube disposed within the outer catheter lumen. Once expandable member 111 is in the expanded configuration, the clinician may operate a medical device to deliver ultrasound energy to the target treatment site via the ultrasound transducer disposed within expandable member 111. The ultrasound energy can, for example, provide or facilitate neuromodulation therapy at the target treatment site. For ease of description, the following discussion will be primarily focused on delivering ultrasound energy. It will be understood, however, that catheter 102 may include elements configured to deliver other types of energy or therapies (e.g., chemical ablation, radiofrequency (RF) ablation, cryoablation, and the like).

FIG. 2 is a conceptual diagram illustrating a schematic cross-sectional view of an example distal portion 108A of catheter 102 of FIG. 1, the cross-section being taken along line A-A in FIG. 1. FIG. 2 also illustrates expandable member 111 in apposition with vessel wall 202 of blood vessel 204. A medical device is configured to delivery ultrasound energy to a target treatment site via ultrasound transducer 218, which is positioned within an inner volume 216 of expandable member 111. In some examples in which blood vessel 204 is a renal vessel, the energy may be used to modulate nerve tissue of the renal plexus adjacent to blood vessel 204 (e.g., by ablating the nerve tissue and creating lesions).

In some instances, catheter 102 may be positioned in a main renal artery, an accessory renal artery, or a branch vessel extending distally from a main renal artery or accessory renal artery. In other examples, catheter 102 may be positioned within another hollow anatomical structure (e.g., a different blood vessel, a non-blood vessel lumen).

Elongated body 108 includes an outer catheter body 205 defining outer catheter lumen 206. As illustrated in FIG. 2, outer catheter lumen 206 is fluidically connected to inner volume 216 of expandable member 111 and facilitates flow of fluid 224 between outer catheter lumen 206 and inner volume 216. Fluid 224 is, for example, an inflation fluid configured to expand expandable member 111 radially outwards into an expanded state (shown in FIG. 2) and a cooling fluid configured to transfer heat away from ultrasound transducer 218.

Catheter 102 further includes inner tube 208 disposed within outer catheter lumen 206 and extending partially into expandable member 111. Inner tube 208 defines inner tube lumen 212. In some examples, an inner member, such as guidewire tube 210 defining guidewire lumen 214 illustrated in FIG. 2, is disposed within inner tube lumen 212. In the example shown in FIG. 2, guidewire tube 210 extends distally past a distal end of inner tube 208 to a distal end 112 of catheter 102. Guidewire tube 210 may be configured to accept a guidewire (not shown in FIG. 2) to facilitate navigation of catheter 102 through the vasculature of the patient. In some examples, as illustrated in FIG. 2, guidewire tube 210 may be connected to distal end 112 of catheter 102 such that an inner member disposed within guidewire lumen 214 may extend distally past distal end 112 and a distal end of the inner member may be disposed at the target treatment site within the patient.

FIG. 2 illustrates expandable member 111 in an expanded configuration and ultrasound transducer 218 positioned within interior volume 216 of expandable member 111. In some examples, ultrasound transducer 218 is separated from an inner surface of expandable member 111 by fluid 224 while expandable member 111 is in the expanded configuration. While expandable member 111 is in the collapsed configuration, the inner surface of expandable member 111 may be in contact with the outer surface of ultrasound transducer 218 or may remain physically separated from ultrasound transducer 218.

Ultrasound transducer 218 includes a distal portion 218A and a proximal portion 218B. Ultrasound transducer 218 includes transducer body 219 defining a transducer lumen 222. Transducer lumen 222 is in fluid communication with inner tube lumen 212, inner volume 216, and outer catheter lumen 206 (through inner volume 216). In some examples, a distal end of inner tube 208 is connected proximal portion 218B of ultrasound transducer 218. For example, a distal end of inner tube 208 can be connected to a proximal end of proximal portion 218B of transducer 218 either directly (e.g., via ultrasonic welding or the like) or indirectly (e.g., via an adhesive, a mechanical connection mechanism, or the like). As a result, a distal end of inner tube lumen 212 is in fluid communication with transducer lumen 222. This fluid communication may be direct in that there is no intermediary volume, other than a structure (e.g., a sleeve) used to mechanically connect inner tube 208 and transducer 218, if present, is positioned between inner tube lumens 212.

In some examples, as illustrated in FIG. 2, a fluid 224 is introduced from inner tube lumen 212 into transducer lumen 222, and then from transducer lumen 222 into inner volume 216 to transform expandable member 111 into the expanded configuration illustrated in FIG. 2. Fluid 224 can then be removed from inner volume 216 through outer catheter lumen 206. This can be performed at the same time as the introduction of fluid 224 into inner volume 216 through inner tube lumen 212 to circulate fluid 224 through expandable member 111 or at a different time. In other examples, fluid 224 is introduced into inner volume 216 through outer catheter lumen 206. The distal end of inner tube 208 may be configured as a support member for proximal portion 218B of ultrasound transducer 218. The distal end of inner tube 208 may support proximal portion 218B of ultrasound transducer 218 by maintain the position and orientation of ultrasound transducer 218 relative to expandable member 111 and/or inner tube 208. Fluid 224 can then be removed from inner volume 216 through inner tube lumen 212. This can be performed at the same time as the introduction of fluid 224 into inner volume 216 through outer catheter lumen 206 to circulate fluid 224 through expandable member 111 or at a different time. In any of these examples, fluid 224 facilitates cooling of ultrasound transducer 218. In addition, in any of these examples, fluid 224 can be automatically introduced and/or removed from inner volume 216 under the control of control circuitry of a medical device or manually introduced under the control of a clinician.

In the example shown in FIG. 2, ultrasound transducer 218 is disposed around longitudinal axis 106 in a geometric shape. For example, transducer body 219 can define an annulus or other geometric shapes such as a geometrical prism. In some examples, transducer body 219 is cylindrical. In some examples, ultrasound transducer 218 is radially disposed a full 360 degrees around longitudinal axis 106 and is configured to transmit ultrasound energy(s) to the tissue of vessel wall 202 in a 360 degree field around longitudinal axis 106. In other examples, ultrasound transducer 218 is disposed less than 360 degrees around longitudinal axis 106. In some examples, a single ultrasound transducer 218 is disposed around longitudinal axis 106. In other examples, multiple ultrasound transducers 218 are disposed around longitudinal axis 106. Multiple ultrasound transducers 218 may be connected together to form the geometric shape.

Ultrasound transducer body 219 can be formed from any suitable material, such as, but not limited to, a piezoelectric material such as a piezoelectric ceramic. The piezoelectric material is configured to vibrate and generate sound waves (e.g., ultrasound waves) in response to an electrical signal being applied across the piezoelectric material. Ultrasound transducer 218 may propagate the sound waves outwards from the inner surface and the outer surface of the transducer body 219 of ultrasound transducer 218.

Ultrasound transducer 218 includes a plurality of electrodes 220 disposed on the outer surface and/or the inner surface of the body of transducer body 219. The plurality of electrodes 220 are configured to transmit an electrical current (e.g., an AC current) from the medical device across transducer body 219. In some examples, as illustrated in FIG. 2, electrodes 220 may be wrapped electrodes configured to wrap around portions of ultrasound transducer 218. The portions can be less than the full outer and inner surface area of transducer body 219. In some examples, electrodes 220 cover a first portion of the outer surface of ultrasound transducer 218 and a second portion of the inner surface of ultrasound transducer 218. Each of the first and second portions can be less than the full surface areas or can cover the full surface areas. In some examples, as illustrated in FIG. 2, the first portion and the second portion least partially overlaps, e.g., in a radial direction. Each of electrodes 220 may be an anode or a cathode. For examples, one or more electrodes 220 on the outer surface of ultrasound transducer 218 may be anodes and one or more electrodes 220 on the inner surface of ultrasound transducer 218 may be cathodes, or vice versa.

Electrodes 220 may be electrically connected to the medical device via a plurality of electrical conductors (not shown) to provide a path for current flow. The conductors may be disposed on or within inner tube 208, outer catheter body 205 of elongated body 108, guidewire tube 210, or the like. For example, in some examples, electrode 220A is electrically connected to an electrically conductive inner tube 208 (or a conductive portion thereof) to provide a path for current flow to ultrasound transducer 218 and electrode 220B is electrically connected to an electrically conductive guidewire tube 210 (or a conductive portion thereof) to provide a return path, or vice versa. Electrically conductive inner tube 208 and/or guidewire tube 210 can eliminate the need for separate electrical conductors, which can allow for increased fluid flow within outer catheter lumen 206 and/or inner tube lumen 212 for a given catheter size. Increased fluid flow within outer catheter lumen 206 and/or inner tube lumen 212 may help more efficiently cool ultrasound transducer 218.

To generate ultrasound energy, a medical device can apply an electrical current from a first electrode 220A to a second electrode 220B through ultrasound transducer 218. For example, first electrode 220A on the first portion of ultrasound transducer 218 may transmit an electrical current to second electrode 220B on the second portion of ultrasound transducer 218 through transducer body 219. In other examples, second electrode 220B may transmit the electrical current to the first electrode 220A through transducer body 219.

Electrodes 220 of ultrasound transducer 218 may establish active region(s) of ultrasound transducer 218. The active region(s) of ultrasound transducer 218 may be areas that are vibrating and generating ultrasound energy. The active region(s) of ultrasound transducer 218 also generate heat from vibrating. In some examples, electrodes 220 and active region(s) may only be positioned on the portions of transducer body 219 where the first portion of the outer surface and the second portion of the inner surface at least partially overlap, e.g., in a radial direction.

When ultrasound transducer 218 vibrates, ultrasound transducer 218 generates heat. The generated heat may cause an increase in the temperature of the ultrasound transducer 218. Ultrasound transducer 218 may transfer a portion of the heat into other objects and/or materials in contact with ultrasound transducer 218 via conduction. In some examples, ultrasound transducer 218 transfers at least a portion of the heat into fluid 224 within transducer lumen 222 and/or inner volume 216. In some examples, ultrasound transducer 218 transfers at least a portion of the heat into transducer support member 221 positioned on distal portion 218A of ultrasound transducer 218.

During operation of ultrasound transducer 218, portions of catheter 102, such as ultrasound transducer 218, overheat due to the buildup of heat, which may lead to loss of function of ultrasound transducer 218. By circulating a cooling fluid 224 around ultrasound transducer 218 (e.g., in an example process as described above), the likelihood of ultrasound transducer 218 overheating may be reduced. The connection between inner tube lumen 212 and transducer lumen 222 facilitates movement of fluid 224 across the inner surface of ultrasound transducer 218 at a faster rate relative to another example catheter where inner tube lumen 212 is not fluidically connected to transducer lumen 222 and/or merely extends through transducer lumen 222 and distally past transducer 218. The connection between inner tube lumen 212 and transducer lumen 222 increases the rate of heat exchange between the inner surface of ultrasound transducer 218 and fluid 224. The increased rate of heat exchange may enhance the cooling effect of fluid 224 on ultrasound transducer 218.

In the example shown in FIG. 2, transducer support member 221 is positioned at distal portion 218A of ultrasound transducer 218. In some examples, catheter 102 includes a single transducer support member 221 positioned at distal portion 218A of ultrasound transducer 218. In some examples, catheter 102 includes two or more transducer support members 221 positioned at distal portion 218A of ultrasound transducer 218. Transducer support member 221 is configured to help maintain the relative positions of ultrasound transducer 218 and guidewire tube 210 within expandable member 111. For example, transducer support member 221 is configured to extend between transducer body 219 and guidewire tube 210 and provide structural support to transducer body 219 to help maintain a radial gap between a more proximal portion of transducer body 219 and guidewire tube 210.

Transducer support member 221 may define an inner member aperture (shown in FIG. 4) (e.g., along longitudinal axis 106) configured to accept an inner member and/or guidewire tube 210. In some examples, catheter 102 may include a plurality of inner members and transducer support member 221 may define one or more inner member apertures configured to accept the plurality of inner members.

In some examples, transducer support member 221 is positioned at portions of ultrasound transducer 218 where the first portion (where first electrode 220A is located) and the second portion (where second electrode 220B is located) do not overlap. In some areas, transducer support member 221 is not placed at portions of ultrasound transducer 218 wherein the first portion and the second portion overlaps.

Transducer support member 221 defines a plurality of fluid apertures (shown in FIG. 4) configured to facilitate movement of fluid 224 between transducer lumen 222 and inner volume 216 of expandable member 111. In some examples, the plurality of fluid apertures may be radially disposed around longitudinal axis 106. Transducer support member 221 may define a single fluid aperture, two fluid apertures, or three or more fluid apertures.

Transducer support member 221 is formed from any suitable material. In some examples, transducer support member 221 is formed from a piezoelectric material (e.g., a piezoelectric ceramic). In some examples, transducer support member 221 is formed from an electrically conductive material that electrically couples piezoelectrical material on the inner surface of transducer body 219 with an electrically conductive guidewire tube 210. In some examples, transducer support member 221 is a single continuous element defining one or more inner member apertures and one or more fluid apertures. In some examples, transducer support members 221 includes a plurality of separate elements which are connected to defining the one or more inner member apertures and the one or more fluid apertures.

In some examples, transducer support member 221 is integral with transducer body 219. In some examples, transducer support member 221 electrically connects guidewire tube 210 to one or more of electrodes 220. In some examples, transducer support member 221 may be separate from and connect to transducer body 219 (e.g., via ultrasound welding, adhesives, or the like). In some, but not all examples, transducer support member 221 is formed from the same material as transducer body 219.

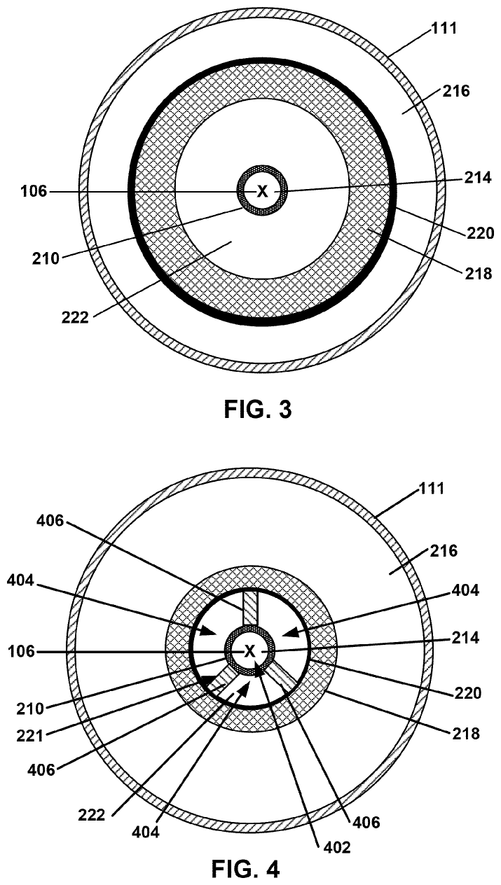

FIG. 3 is a conceptual diagram illustrating a schematic cross-sectional view of the example distal portion 108A of catheter 102 of FIG. 2, the cross-section being taken along line B-B in FIG. 2. As illustrated, the cross-section is taken along a plane orthogonal to longitudinal axis 106 and at a proximal portion 218B of ultrasound transducer 218. FIG. 3 illustrates ultrasound transducer 218 disposed within inner volume 216 of expandable member 111.

As discussed above, ultrasound transducer 218 includes electrode(s) 220 disposed on at least a portion of ultrasound transducer 218. In some examples, as illustrated in FIG. 3, electrode(s) 220 are disposed on at least a portion of the outer surface of ultrasound transducer 218 (e.g., on a first portion of the outer surface of ultrasound transducer 218). In some examples, electrode(s) 220 are also disposed on at least a portion of the inner surface of ultrasound transducer 218 (e.g., on a second portion of the inner surface of ultrasound transducer 218). Guidewire tube 210 defining guidewire lumen 214 may be disposed within transducer lumen 222 and may be configured to accept an inner member (e.g., a guidewire).

In some examples, as illustrated in FIG. 3, ultrasound transducer 218 is unsupported at proximal portion 218B of ultrasound transducer 218. For example, in some examples, the inner surface of transducer body 219 of ultrasound transducer 218 is not connected to any other portion of catheter 102 (e.g., to transducer support member 221 (shown in FIG. 2), to guidewire tube 210, or the like). In some examples, ultrasound transducer 218 is supported at a proximal end of proximal portion 218B of ultrasound transducer 218 by inner tube 208 and may be unsupported for a length (measured along longitudinal axis 106) of ultrasound transducer 218 distal to the proximal end of proximal portion 218B. Portions of ultrasound transducer 218 may be unsupported to reduce the transfer of heat into any transducer support member 221 and/or guidewire tube 210, which may lead to heat buildup in transducer support member 221 and/or guidewire tube 210. Instead, unsupported portions of ultrasound transducer 218 enable transmission of heat directly into fluid 224 (shown in FIG. 2) flowing within transducer lumen 222. The transmission of the heat directly into fluid 224 may facilitate efficient cooling of ultrasound transducer 218 and reduce heat buildup within ultrasound transducer 218.

FIG. 4 is a conceptual diagram illustrating a schematic cross-sectional view of the example distal portion 108A of catheter 102 of FIG. 2, the cross-section being taken along line C-C in FIG. 2. As illustrated, the cross-section is taken along a plane orthogonal to longitudinal axis 106 and at a distal portion 218A of ultrasound transducer 218. FIG. 4 illustrates ultrasound transducer 218 is disposed within inner volume 216 of expandable member 111. A distal portion of ultrasound transducer 218 is supported by transducer support member 221. Transducer support member 221 is positioned between ultrasound transducer 218 and guidewire tube 210, and helps to maintain the relative position of ultrasound transducer 218 and guidewire tube 210 within inner volume 216 of expandable member 111.

In some examples, as illustrated in FIG. 4, transducer support member 221 includes a plurality of support arms 406 disposed radially around longitudinal axis 106 and connecting ultrasound transducer 218 and guidewire tube 210. In some examples, for a given transducer support member 221 outer diameter of 1.2 mm to 1.6 mm and a guide tube 210 diameter of 0.4 mm to about 0.6 mm (e.g., 0.5 mm), each of support arms 406 has a length of about 0.3 millimeters (mm) to about 0.6 mm, such as 0.35 mm to 0.55 mm. The plurality of transducer support arms 406 obscure a portion of a cross-sectional area of transducer lumen 222 and fluid apertures 404 of support member 221 are defined between support arms 406 to enable fluid flow through support member 221 (e.g., in a proximal to distal direction). In some examples, the plurality of transducer support arms 406 obscures up to about 60% of the cross-sectional area, such as about 50% or less of the cross-sectional area. The portion of the cross-sectional area obscured by the plurality of transducer support arms 406 may be selected based on a maximum allowable pressure for flow of fluid 224 within expandable member 111. Transducer support member 221 also defines inner member aperture 402 configured to receive guidewire tube 210 (or another inner member). In some examples, as illustrated in FIG. 4, transducer support member 221 includes three support arms 406. In other examples, transducer support member 221 includes two, four, five, or more support arms 406, or a different configuration defining fluid apertures 404 and inner member aperture 402.

Transducer support arms 406 may be evenly or unevenly distributed. While the example catheter 102 of FIGS. 2 and 4 illustrate transducer support member 221 disposed on a distal portion 218A of ultrasound transducer 218, transducer support member 221 may be disposed on other portions of ultrasound transducer 218 (e.g., at a proximal portion 218B and/or at a medial portion of ultrasound transducer 218).

In addition, although the example catheter 102 includes one transducer support member 221, in other examples, catheter 102 can include two or more transducer support member 221 that are longitudinally displaced from each other along longitudinal axis 106. The inner member apertures 402 and fluid apertures 404 of the two or more transducer support members 221 can be aligned to facilitate fluid flow in a direction along longitudinal axis 106 past the support members 221.

In some examples, as illustrated in FIG. 4, the one or more transducer support members 221 are disposed on a portion of distal portion 218A of ultrasound transducer 218 that is an inactive area. Transducer support members 221 on an inactive area to reduce transmission of heat into one or more of transducer support members 221 and lead to heat buildup within the one or more transducer support members 221. Active areas of ultrasound transducer 218 may first transmit the heat into inactive areas of ultrasound transducer 218 and the inactive areas of ultrasound transducer 218 may then transmit heat into the one or more transducer support members 221. Fluid 224 may flow around transducer support members 221 through fluid aperture(s) 404 and may cool transducer support members 221 by facilitating the transfer of heat from transducer support members 221 to fluid 224 in fluid apertures 404.

In some examples, inner member aperture(s) 402 may be sized to fit snugly with guidewire tube 210. In some examples, inner member aperture(s) 402 may be configured to enable attachment of transducer support member 221 to guidewire tube 210.

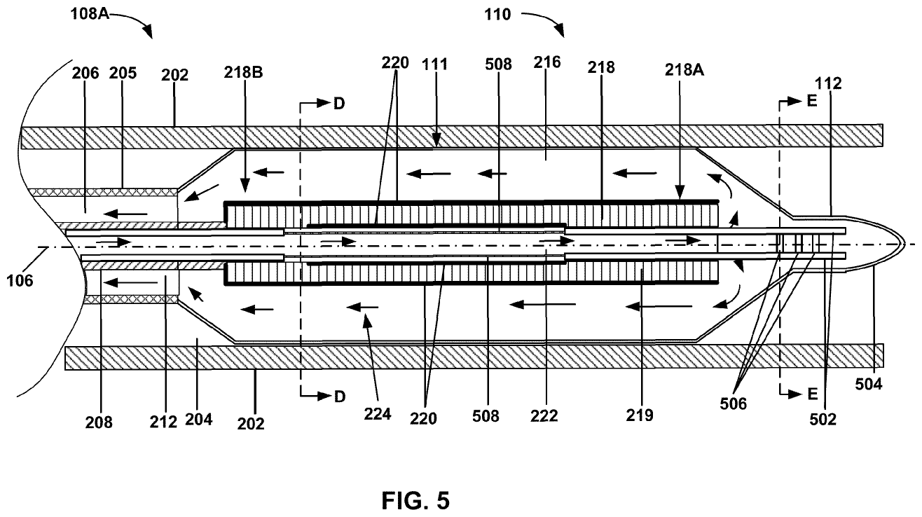

FIG. 5 is a conceptual diagram illustrating a schematic cross-sectional view of another example distal portion 108A of catheter 102 of FIG. 1, the cross-section being taken along line A-A in FIG. 1. In contrast to the example of distal portion 108A shown in FIGS. 2-4, the example shown in FIGS. 5-7 illustrates an example distal portion 108A including a plurality of guide tubes 502 and support member 221 positioned distal to ultrasound transducer 218.

The example distal portion 108A illustrated in FIG. 5 includes a plurality of guide tubes 502 disposed within transducer lumen 222, each of the plurality of guide tubes 502 configured to accept an inner member (e.g., a navigation wire, a push/pull wire 508, or the like). The inner member(s) is configured to help steer distal portion 108A through vasculature of patient (e.g., by deflecting distal portion 108A in one or more directions away from longitudinal axis 106). The example distal portion 108A of FIG. 5 can eliminate the need for guidewire tube 210 within transducer lumen 222 and may allow for a larger volume of fluid 224 be delivered through outer catheter lumen 206 and into transducer lumen 222 for a given outer diameter outer catheter body 205 and ultrasound transducer 218. The larger volume of fluid 224 delivered into transducer lumen 222 may help facilitate more efficient heat transfer away from transducer body 219 to cool transducer body 219. In some examples, the plurality of guide tubes 502 enables distal end 112 of catheter 102 to be closed.

Guide tubes 502 may be disposed within transducer lumen 222 and on the inner surface of ultrasound transducer 218. In some examples, guide tubes 502 may be positioned radially inwards of, e.g., disposed on, electrode(s) 220 disposed on the inner surface of ultrasound transducer 218. Each of guide tubes 502 may be a tubular body that defines an inner member tube lumen (not shown in FIG. 5) configured to contain a push/pull wire 508. In some examples, each of guide tubes 502 is a single continuous element extending from a proximal portion 108B to distal end 112 of catheter 102. In other examples, as illustrated in FIG. 5, one or more (e.g., all) of the guide tubes 502 includes a plurality of segments. For each of guide tubes 502, a first segment may be connected to and/or support proximal portion 218B of ultrasound transducer 218 and a second segment may be connected to and/or support distal portion 218A of ultrasound transducer 218. In some examples, the first segment and the second segment of each of guide tubes 502 may be separated, e.g., at active area(s) of ultrasound transducer 218. The separations in guide tubes 502 may reduce the transfer of heat into guide tubes 502 from ultrasound transducer 218 and/or facilitate the cooling of ultrasound transducer 218. While the example catheter 102 illustrated in distal portion 108A includes two guide tubes 502, other example catheters may include a single guide tube 502 or three or more guide tubes 502. In some examples, guide tubes 502 may be positioned 90 degrees or 180 degrees apart.

Guide tubes 502 are supported by one or more transducer support member 506. For example, each support member 506 extends between guide tubes 502. Each support member 506 is configured to help maintain the relative position and/or orientation of guide tubes 502 and ultrasound transducer 218 within inner volume 216 of expandable member 111. In some examples, as illustrated in FIG. 5, some or all of the support members 506 are disposed distal of ultrasound transducer 218. In other examples, support member 506 is disposed within transducer lumen 222 (e.g., at distal portion 218A of ultrasound transducer 218, similar to support member 221 of FIG. 4). In some examples, support member 506 defines fluid apertures configured to facilitate the flow of fluid 224 through support member 506. While FIG. 5 illustrates distal portion 108A as having three support members 506, other example distal portions 108A may include a single support member 506, two support members 506, and four or more support members 506.

Push/pull wires 508 are disposed within guide tubes 502. A clinician may navigate catheter 102 through the vasculature of the patient by exerting a pulling and/or pushing force on one or more of push/pull wires 508, which causes steerable tip 504 of catheter 102 to deflect in a particular direction.

Steerable tip 504 is configured to facilitate navigation of distal portion 108A of catheter 102 through vasculature of the patient. Steerable tip 504 is tapered in a distal direction relative therapeutic element 110. Steerable tip 504 may be relatively flexible to facilitate deflection of distal portion 108A away from longitudinal axis 106 in one or more directions. Steerable tip 504 may be made of a biocompatible polymer. The polymer may include, for example, a thermoplastic, such as an elastomer. In some examples, the elastomer may include a polyurethane, a silicone, or a copolymer, such as a block copolymer including polyether block amide available under the trade name Pebax® available from Arkema S.A., Colombes, France. In some examples, steerable tip 504 may be formed from a polymer with a relatively low Shore hardness so steerable tip 504 presents a relatively atraumatic tip in case of contact between steerable tip 504 and tissue.

In some examples, steerable tip 504 may have a relatively extended longitudinal length. In some examples, the steerable tip 504 has a relatively extended longitudinal length of up to about 30 mm, such as 5 mm to 30 mm. This may facilitate advancing of distal portion 108A of catheter 102 through vasculature of a patient, e.g., in the absence of a guidewire. In some examples, the length of steerable tip 504, measured parallel to longitudinal axis 106, may be between about 5 mm and about 2 centimeters (cm).

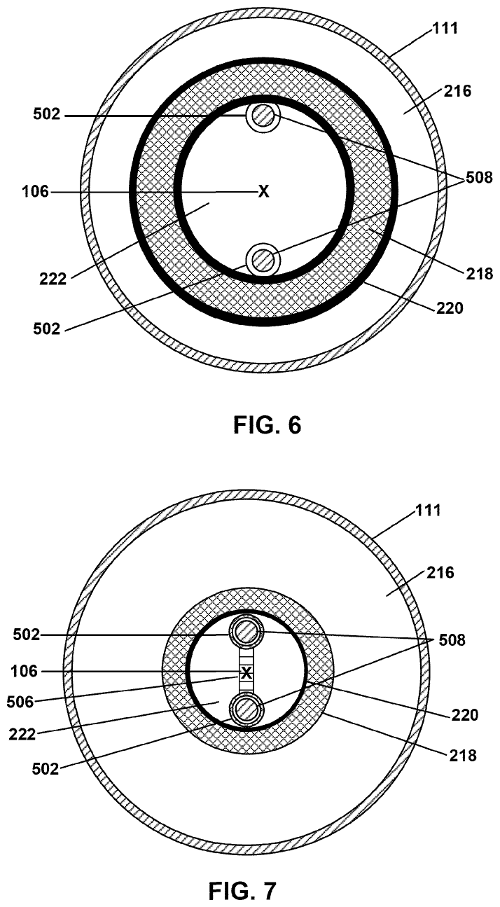

FIG. 6 is a conceptual diagram illustrating a cross-sectional view of the example distal portion 108A of catheter 102 of FIG. 5, the cross-section being taken along line D-D in FIG. 5. The cross-section is taken along a plane orthogonal to longitudinal axis 106 and at a proximal portion 218B of ultrasound transducer 218. FIG. 6 illustrates ultrasound transducer 218 disposed within inner volume of expandable member 111, and guide tubes 502 and push/pull wires 508 may be disposed within transducer lumen 222.

In some examples, guide tubes 502 are coincident to and/or attached to an inner surface of ultrasound transducer 218. Guide tubes 502 may support position of ultrasound transducer 218. Guide tubes 502 may be coincident to ultrasound transducer 218 at inactive areas of ultrasound transducer 218. As illustrated in FIGS. 5 and 6, portions of push/pull wires 508 may be exposed to fluid 224 within transducer lumen 222. When exposed to fluid 224, push/pull wires 508 may be separated from the inner surface of ultrasound transducer 218 by fluid 224.

FIG. 7 is a conceptual diagram illustrating a schematic cross-sectional view of the example distal portion 108A of catheter 102 of FIG. 5, the cross-section being taken along line E-E in FIG. 5. The cross-section is taken along a plane orthogonal to longitudinal axis 106 and at a distal portion 218A of ultrasound transducer 218. As illustrated, guide tubes 502 may be coincident to and/or attached to the inner surface of ultrasound transducer 218. In some examples, as illustrated in FIG. 7, guide tubes 502 are at least partially co-extensive with electrode(s) 220 disposed on the inner surface of ultrasound transducer 218. Transducer support member 506 may connect the outer surfaces of guide tubes 502 such that each of guide tubes 502 maintain position and/or orientation relative to another guide tube 502 and/or ultrasound transducer 218.

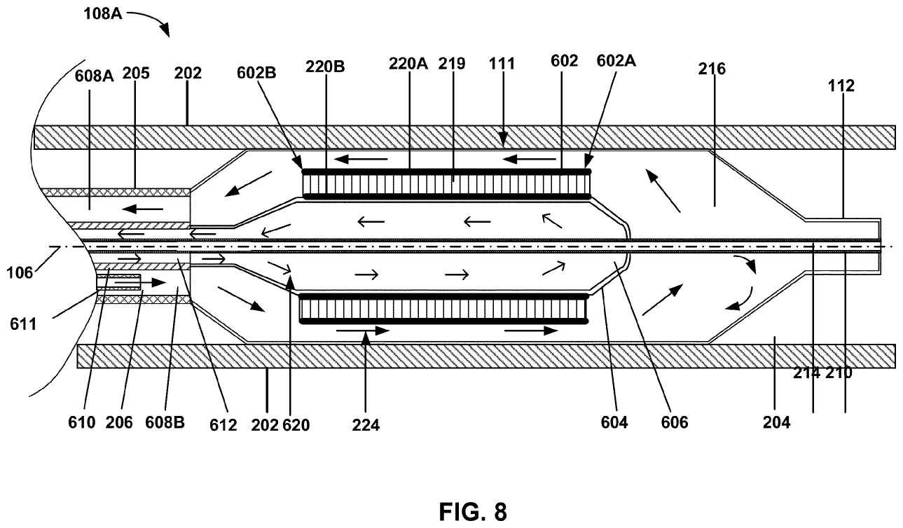

FIG. 8 is a conceptual diagram illustrating a schematic cross-sectional view of another example distal portion 108A of catheter 102 of FIG. 1, the cross-section being taken along line A-A in FIG. 1. In contrast to the examples of distal portion 108A shown in FIGS. 1-7, the example shown in FIG. 8 illustrates another example distal portion 108A with a second expandable member 604 disposed within expandable member 111. Second expandable member 604 is disposed radially inward of ultrasound transducer 602. For example, ultrasound transducer 602 can be disposed on an outer surface of second expandable member 604. Second expandable member 604 may be filled with a second medium 620 configured to impede propagation of ultrasound energy, which can help cool both ultrasound transducer 602 and guidewire tube 210 (or other inner member that extends through second expandable member 604).

As discussed above, elongated body 108 includes an outer catheter body 205 defining outer catheter lumen 206. Outer catheter lumen 206 is fluidically connected to inner volume 216 of (outer) expandable member 111. In the example shown in FIG. 8, fluid delivery tube 611 is disposed within outer catheter lumen 206. Fluid delivery tube 611 is configured to deliver fluid 224 into inner volume 216 of expandable member 111. Fluid 224 can be introduced into inner volume 216 via fluid delivery tube 611 and removed from inner volume 216 via outer catheter lumen 206. This may enable expansion of expandable member 111, deflation of expandable member 111, and/or circulation of fluid 224 within inner volume 216, e.g., to cool ultrasound transducer 602.

Fluid delivery tube 611 has any suitable configuration and can be, for example, a tubular body configured to extend through outer catheter lumen 206 from a proximal fluid source to expandable member 111. Although shown FIG. 8 as terminating proximal to a proximal end of expandable member 111, in other examples, fluid delivery tube 611 extends distally into inner volume 216 of expandable member 111.

Second expandable member 604 is configured to receive, e.g., to transition to an expanded state from a less expanded state, a second medium 620 within an inner volume 606 of second expandable member 604. In some examples, second expandable member 604 may be a balloon, e.g., a compliant balloon. In some examples, second expandable member 604 may be expanded while expandable member 111 is in the collapsed configuration. Elongated body 108 includes an inner tube 610 disposed within outer catheter lumen 206, inner tube 610 defining an inner tube lumen 612 configured to deliver second medium 620 to inner volume 606. The clinician may introduce and/or remove second medium 620 from inner volume 606 of second expandable member 604 using inner tube lumen 612. For example, the clinician can introduce second medium 620 into inner volume 606 via inner tube lumen 612 to expand second expandable member 604, or remove second medium 620 from inner volume 606 via inner tube lumen 612 to deflate second expandable member 604. In some examples, the clinician may circulate second medium 620 within inner volume 606, e.g., to facilitate cooling of ultrasound transducer 218. In these examples, another inner tube like inner tube 610 can be disposed within outer catheter lumen 206, and one inner tube 610 can be used to deliver fluid into inner volume 606 and the other inner tube can be used to remove the fluid from inner volume 606.

In some examples, second medium 620 is a medium that impedes the transmission of sound energy, e.g., ultrasound energy. For example, second medium 620 may impede the transmission of sound energy from ultrasound transducer 602 into second expandable member 604. In these examples, second medium 620 may not heat-up as much as fluid 224 during operation of ultrasound transducer 602, and, therefore, may not need to be cycled through inner volume 606 of second expandable member 604 during operation of ultrasound transducer 602 or at least not cycled as much. Instead, for example, second medium 620 may be delivered into inner volume 606 and held in the inner volume 606 until the end of the medical procedure, at which time second medium 620 can be removed from inner volume 606. By impeding the transmission of sound energy into second expandable member 604, second medium 620 may reduce the rate of heating of ultrasound transducer 602, second expandable member 604, guidewire tube 210, or the like. Second medium 620 may be a gas including air, nitrous oxide, carbon dioxide, helium, or the like.

In some examples, as illustrated in FIG. 8, electrode(s) 220 are disposed on the outer surface and the inner surface of ultrasound transducer 602 to define active area(s) (also referred to as “active transducer area(s)”) of ultrasound transducer 602. In some examples, ultrasound transducer 602 defines the active area(s) in portions of ultrasound transducer where electrode(s) 220 disposed on the outer surface and the inner surface of ultrasound transducer 602 overlap. In some examples, due to the properties of second medium 620, ultrasound transducer 602 may not transmit energy to the inner surface of ultrasound transducer 602 when vibrating and may reduce heat buildup in ultrasound transducer 602. In some examples, as illustrated in FIG. 8, electrode(s) 220 may not be wrapped around ultrasound transducer body 219. In some examples, electrode(s) 220 may be wrapped around ultrasound transducer body 219 and may be disposed on the outer surface and the inner surface of ultrasound transducer 602 (e.g., of ultrasound transducer body 219), e.g., in a manner substantially similar to electrode(s) 220 illustrated in FIG. 2.

Second expandable member 604 may include an electrically conductive material configured to electrically connect electrode(s) 220 of ultrasound transducer 602 with a medical device proximal to catheter 102. The electrically conductive material may include a metallic foil.

Guidewire tube 210 may be disposed within inner tube lumen 612, second expandable member 604, and expandable member 111. Guidewire tube 210 defines a guidewire lumen 214 configured to accept an inner member, (e.g., a guidewire, a push/pull wire, an outer sheath, another catheter, or the like). In some examples, distal end 112 of catheter 102 may include a steerable tip (e.g., steerable tip 504 of FIG. 5). In some examples, guidewire tube 210 is electrically connected to electrode(s) 220 via second expandable member 604. In some examples, an electrically conductive surface of second expandable member 604 may be electrically connected to electrodes 220 while electrode 220 are in a wrapped configuration. A distal portion of second expandable member 604 may be electrically connected to electrode 220B and a proximal portion may be electrically connected to a wrapped portion of electrode 220A or vice versa. In some examples, second expandable member 604 (e.g., a proximal portion of second expandable member 604) forms an electrically conductive path from inner tube 208 to electrode 220A. In some examples, electrode(s) 220 are wrapped around an outer surface of second expandable member 604 and is configured to fold with second expandable member 604 when second expandable member 604 is deflated.

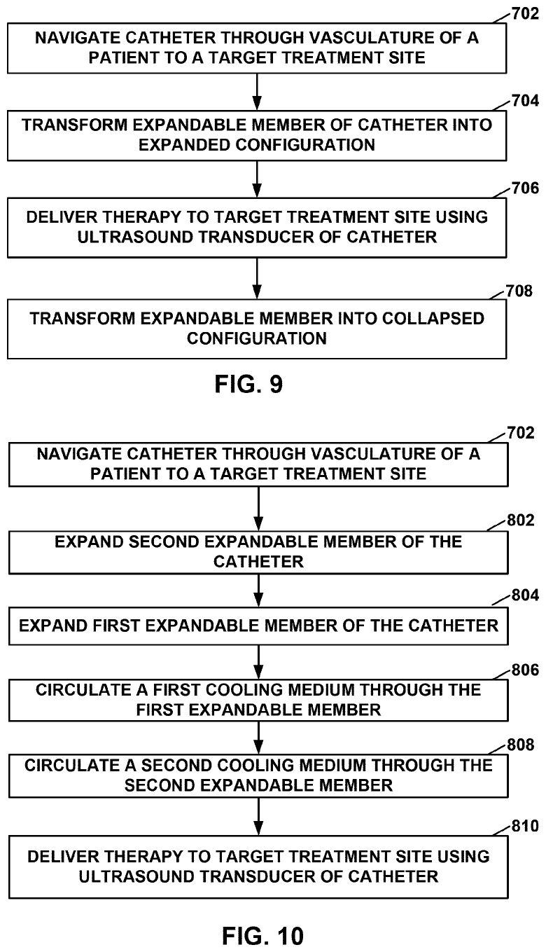

FIG. 9 is a flow diagram illustrating an example process of delivering ultrasound therapy to a patient using a catheter (e.g., catheter 102) that includes an expandable member (e.g., expandable member 111).

A clinician navigates catheter 102 through vasculature of a patient to a target treatment site (702). The clinician may navigate catheter 102 from an access site (e.g., in a femoral artery, a brachial artery, a radial artery, or the like) through blood vessels using a guide member (e.g., a guidewire, a push/pull wire, an outer sheath, another catheter, or the like). In some examples, the clinician may use over-the-wire (OTW) or rapid exchange (RX) techniques to navigate catheter 102 to the target treatment site. In some examples, the clinician may use a radiopaque marker positioned on catheter 102 (e.g., on distal end 112 of catheter 102) to determine the position of distal portion 108A of catheter 102 within vasculature of patient, e.g., through fluoroscopy, magnetic resonance imaging (MRI), or the like.

The target treatment site may be any location at which the clinician intends to deliver ultrasound energy to vessel wall 202 (FIG. 2). In some examples, the clinician may select a plurality of target treatment sites for treatment. When the clinician navigates catheter 102 to the target treatment site, catheter 102 is configured in a relatively low profile delivery configuration, such as when expandable member 111 is in a collapsed configuration.

The clinician may transform expandable member 111 of catheter 102 into an expanded configuration (704). For example, the clinician may introduce a fluid (e.g., fluid 224) into an inner volume (e.g., inner volume 216) of expandable member 111 to expand expandable member 111 into the expanded configuration. In some examples, the clinician may cause a medical device fluidically connected to inner volume 216 of expandable member 111 to introduce fluid 224 into inner volume 216. The clinician may introduce fluid 224 into catheter 102 through a port on catheter 102. The port on catheter 102 may be, for example, proximal to handle 104 of catheter 102. Fluid 224 may flow into inner volume 216 though an outer catheter lumen 206 within elongated body 108. In some examples, fluid 224 enters inner tube lumen 212 within inner tube 208 and travels within elongated body 108 to therapeutic element 110. Fluid 224 then flows from inner tube lumen 212 into transducer lumen 222 defined by ultrasound transducer 218. Fluid 224 flows from transducer lumen 222 into inner volume 216 through one or more of fluid apertures 404 and into inner volume 216 of expandable member 111. Fluid 224 then exits inner volume 216 though outer catheter lumen 206 and exits catheter 102 through a port in catheter 102, e.g., located proximal to handle 104. In some examples, depending on the size of blood vessel 204 at the target treatment site, expandable member 111 may be expanded such that the outer surface of expandable member 111 is in apposition with vessel wall 202 of blood vessel 204. In some examples, the target treatment site may be adjacent to renal nerve(s) for renal denervation therapies. Other target treatment sites are contemplated, such as in other arteries (e.g., a hepatic artery) or in other hollow anatomical structures within the patient.

The clinician delivers an ultrasound energy to target treatment site using ultrasound transducer 218 of catheter 102 (706). For example, the clinician may operate a medical device electrically connected to catheter 102 to transmit an electrical current, e.g., an AC current, to one or more electrodes 220 of ultrasound transducer 218 via one or more electrical conductors disposed on or within elongated body 108 of catheter 102. On at least some portions of ultrasound transducer 218, electrode(s) 220 on a first surface of ultrasound transducer body 219 may transmit the electrical current through the body of ultrasound transducer body 219 to electrode 220 on a second surface of ultrasound transducer body 219. The first surface may be an outer surface of ultrasound transducer body 219 and the second surface may be an inner surface of ultrasound transducer body 219, or vice versa.

Transducer body 219 of ultrasound transducer 218 may include a piezoelectric material (e.g., a piezoelectric ceramic or the like). In response to the electrical current transmitted by electrode(s) 220, ultrasound transducer 218 may begin vibrating and generating sound energy (e.g., ultrasound energy, acoustic energy, or the like). Ultrasound transducer 218 may transmit the sound energy through fluid 224 and into the tissue of the patient at the target treatment site. Ultrasound transducer 218 may transmit the sound energy radially outwards and/or radially inwards of transducer body 219.

The clinician may, as part of delivering the ultrasound energy to the patient, circulate fluid 224 within inner volume 216 of expandable member 111. Ultrasound transducer 218 may generate heat as a byproduct of generating the sound energy. The clinician may circulate fluid 224 within expandable member 111 to cool ultrasound transducer 218 and prevent overheating of ultrasound transducer 218. In some examples, the clinician may circulate fluid 224 within expandable member 111 by transmitting fluid 224 into expandable member 111 via an inner tube lumen (e.g., inner tube lumen 212) of an inner tube (e.g., inner tube 208) within outer catheter lumen 206 of elongated body 108.

The clinician may transform expandable member 111 back into collapsed configuration (708). In some examples, the clinician may transform expandable member 111 after delivering the ultrasound energy to tissue at the target treatment site. The clinician may collapse expandable member 111 by retracting fluid 224 from inner volume 216 of expandable member 111, e.g., via outer catheter lumen 206. After transforming expandable member 111 into the collapsed configuration, the clinician may retract catheter 102 from the vasculature of the patient. In some examples, the clinician may navigate catheter 102 to another target treatment site within the patient.

FIG. 10 is a flow diagram illustrating another example process of delivering ultrasound therapy to a patient using another catheter 102 that includes an expandable member 111. The example catheter 102 may include a second expandable member 604 (FIG. 8) disposed within inner volume 216 of expandable member 111.

A clinician navigates catheter through vasculature of a patient to a target treatment site (702) and expands second expandable member 604 of catheter 102 (802). In some examples, the clinician may expand second expandable member 604 prior to inserting distal portion 108A of catheter 102 into the patient. In other examples, the clinician may expand second expandable member 604 when distal portion 108A is within the vasculature of the patient. The clinician may expand second expandable member 604 by introducing a second medium 620 into second expandable member 604 via an inner tube lumen 612 of inner tube 610.

The clinician may transform expandable member 111 of catheter 102 into expanded configuration (804). The clinician may delivery therapy to target treatment site using ultrasound transducer 218 of catheter 102 (806). The clinician may transform expandable member 111 into collapsed configuration (808). In some examples, the clinician may collapse second expandable member 604 prior to retracting catheter 102 and/or navigating catheter 102 to a new target treatment site.

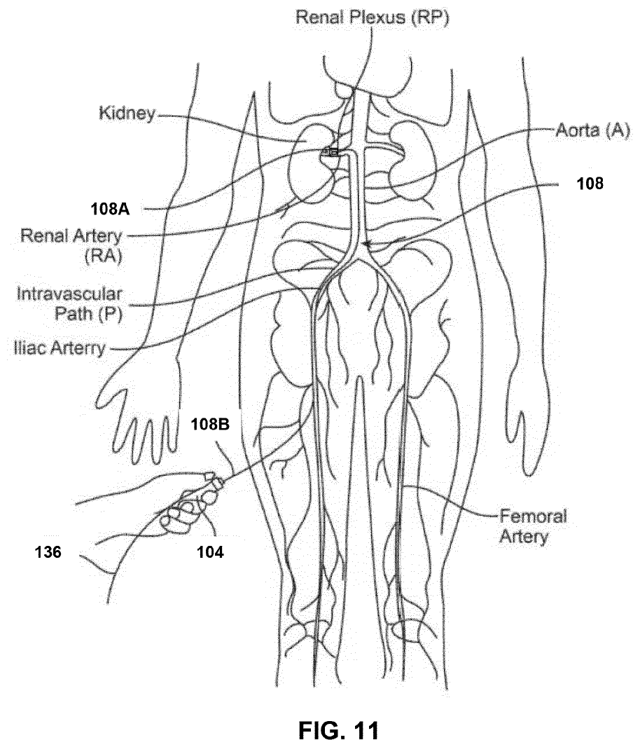

As discussed above, in some examples, catheter 102 may be used to access and modulate renal nerves through renal denervation. FIGS. 11-17 illustrates human anatomy relevant to renal denervation and example techniques for modulating renal nerves with catheter 102. In other examples, catheter 102 may be used with other medical procedures.

FIG. 11 illustrates an example technique for accessing a renal artery and modulating renal nerves with the catheter 102 of FIG. 1. While FIG. 11 illustrates the use of catheter 102 for renal neuromodulation, catheter 102 may be used for other therapies and treatments within another blood vessel or other hollow anatomical body within the human body. Catheter 102 is configured to delivery energy (e.g., radiofrequency energy, ultrasound energy, electrical stimulation energy, or the like) to one or more target treatment sites within a renal vessel. Catheter 102 provides access to the renal plexus (RP) through an intravascular path (P), such as a percutaneous access site in the femoral (illustrated), brachial, radial, or axillary artery to the target treatment sites within a respective renal artery (RA). By manipulating proximal portion 108B of elongated body 108 from outside the intravascular path (P), a clinician may advance at least distal portion 108A of elongated body 108 through the sometimes-tortuous intravascular path (P) and remotely manipulate distal portion 108A (FIG. 1) of elongated body 108. Distal portion 108A may be remotely manipulated by the clinician using the handle 104.

In the example illustrated in FIG. 11, distal portion 108A is delivered intravascularly to the treatment site using an inner member 136 in an over-the-wire (OTW) technique. Inner member 136 may be internal to catheter 102 (e.g., a guide wire, inner catheter, or the like) or external to catheter 102 (e.g., an outer sheath or the like). In some examples, inner member 136 is a navigation wire. Catheter 102 may define a passageway for receiving inner member 136 for delivery of catheter 102 using either an OTW or a rapid-exchange (RX) technique. At the treatment site, inner member 136 can be at least partially withdrawn or removed relative to catheter 102 and distal portion 108A can transform into an expanded configuration (e.g., a helical configuration, a spiral configuration, or the like) for delivering ultrasound energy. In other examples, elongated body 108 may be self-steerable such that therapeutic element 110 may be delivered to the target treatment site without the aid of inner member 136.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced or induced in another suitable manner through the delivery of energy (RF energy, ultrasound energy, microwave energy, or the like). The target treatment site can be within or otherwise proximate to a renal lumen (e.g., a renal artery, a ureter, a renal pelvis, a major renal calyx, a minor renal calyx, or another suitable structure), and the target treatment site can include tissue at least proximate to a wall of the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of the target treatment devices and associate methods for achieving renal neuromodulation via intravascular access and impose specific design requirements for such devices. Specific design requirements may include accessing the renal artery, positioning distal portion 108A within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operated through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic neurons).

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

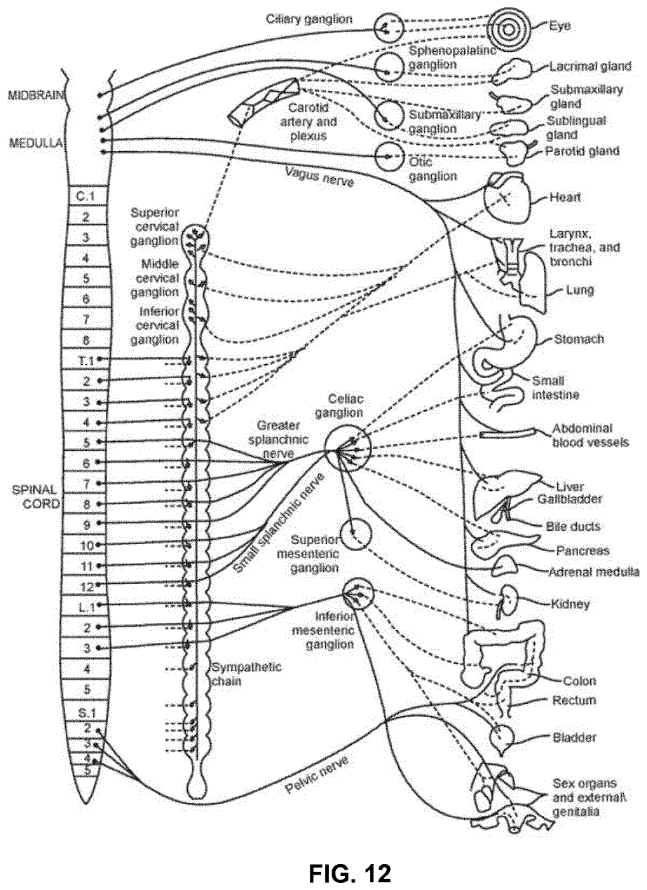

FIG. 12 is a conceptual illustration of an example sympathetic nervous system (SNS) illustrating how the brain communicates with the body via the SNS. As shown in FIG. 12, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of sympathetic nerves leave the spinal cord through the anterior rootlet/root. The axons pass near the spinal (sensory) ganglion, where the axons enter the anterior rami of the spinal nerves. However, unlike somatic innervation, the axons separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

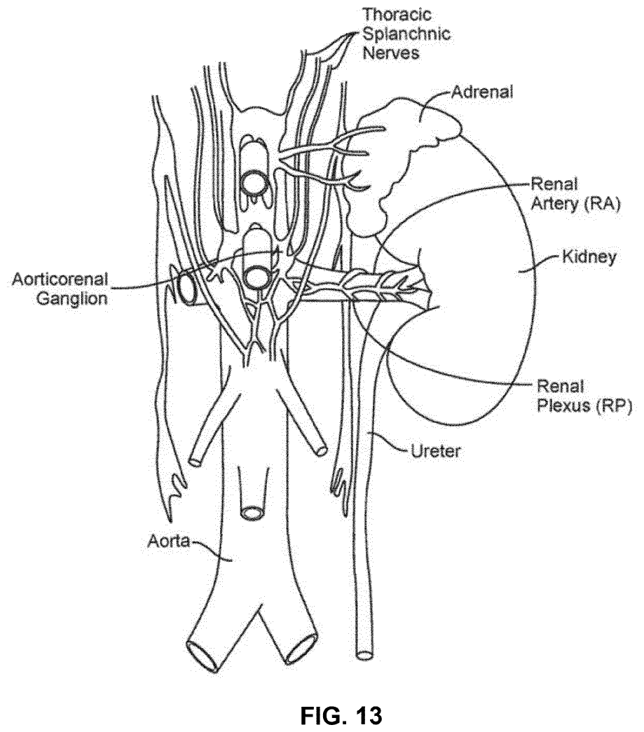

FIG. 13 is an enlarged anatomic view of nerves innervating a left kidney to form the renal plexus surrounding the left renal artery. As FIG. 13 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bi-directional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of theses disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration late, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the media have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus, and the renal tubules. Stimulation of the renal sympathetic nerves cause increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication.

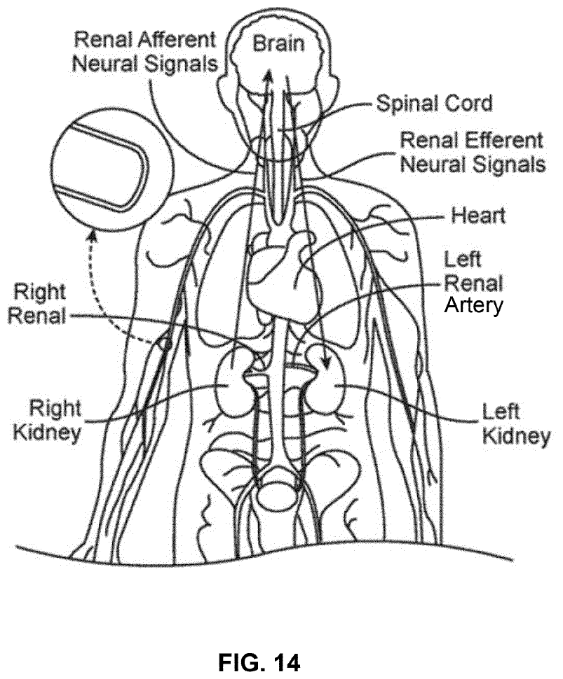

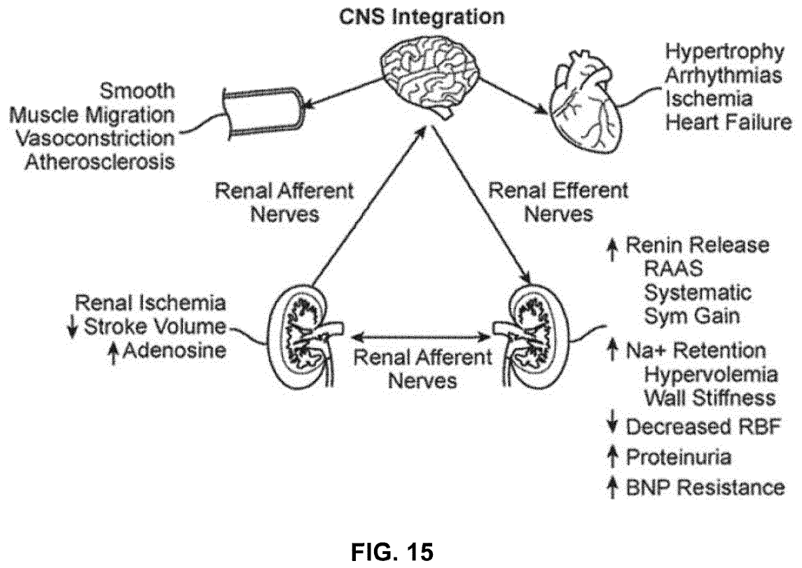

FIG. 14 is an anatomic view of a human body depicting neural efferent and afferent communication between the brain and kidneys. FIG. 15 is a conceptual view of a human body depicting neural efferent and afferent communication between the brain and kidneys. As shown in FIGS. 14 and 15, the afferent communication might be from kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome and sudden death. Since the reduction of afferent neural signals contributing to the systemic reduction of sympathetic tone/drive, renal denervation might also be useful in treating other conditions associate with systemic sympathetic hyperactivity. Accordingly, renal denervation may also benefit other organs and bodily structures innervated by sympathetic nerves, including those identified in FIG. 14. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

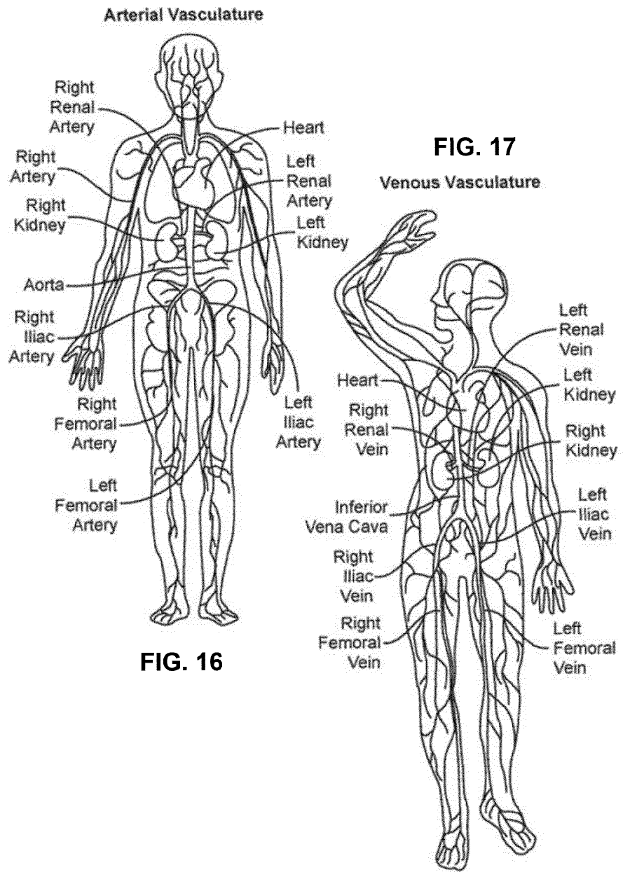

In accordance with the present technology neuromodulation of a left and/or right renal plexus (RP), which is intimately associated with a left and/or right renal artery, may be achieved through intravascular access. FIG. 16 is an anatomic view of the arterial vasculature of a human. As FIG. 16 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

FIG. 17 is an anatomic view of the venous vasculature of a human. As FIG. 17 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base on the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters (e.g., catheter 102) introduced via these access points may be passed through the subclavian artery on the left side (or via the subclavian and brachiocephalic arteries on the right side), through the aortic arch, down the descending aorta and into the renal arteries using standard angiographic techniques. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems, and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of distal portion 108A and therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable distal portion 108A of catheter 102 may intimately contact the vessel wall and/or extend at least partially through the vessel wall. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Various aspects of the disclosure have been described, such as in the following examples. These and other aspects are within the scope of the claims.

Example 1: A catheter comprising: an expandable member; an outer catheter body comprising an outer catheter lumen in fluid communication with the expandable member; an inner tube positioned within the outer catheter lumen, the inner tube defining an inner tube lumen configured to deliver a fluid to the expandable member; and an ultrasound transducer positioned in the expandable member, the transducer comprising: a transducer body comprising an inner surface defining a transducer lumen, wherein at least a proximal portion of the transducer body is connected to a distal portion of the inner tube to fluidically connect the inner tube lumen and the transducer lumen; a transducer support member positioned at a distal portion of the transducer body, the transducer support member defining one or more fluid apertures through which the fluid is configured to flow from the transducer lumen into the expandable member; and a plurality of electrodes extending along at least a first portion of an outer surface of the transducer body and at least a second portion of the inner surface of the transducer body.

Example 2: The catheter of example 1, wherein the first portion of the outer surface and the second portion of the inner surface at least partially overlap in a radial direction.

Example 3: The catheter of example 1 or example 2, further comprising a guidewire tube disposed within the inner tube lumen and the transducer lumen, and extending through the expandable member, the guidewire tube defining a guidewire lumen.

Example 4: The catheter of example 3, wherein the transducer support member electrically connects the plurality of electrodes to the guidewire tube.

Example 5: The catheter of example 3 or example 4, wherein the transducer support member is positioned between distal portion of the transducer body and the guidewire tube in a radial direction, and wherein the plurality of electrodes wrap around a proximal end of the transducer body.

Example 6: The catheter of any of examples 1-5, wherein the plurality of electrodes define an active transducer area.

Example 7: The catheter of any of examples 1-6, further comprising a steerable tip at a distal end of the catheter.

Example 8: The catheter of example 7, wherein the catheter does not include a guidewire lumen.

Example 9: The catheter of examples 7 or 8, further comprising a navigation wire configured to cause the catheter to deflect.

Example 10: The catheter of example 9, wherein the navigation wire extends through the inner tube lumen and the transducer lumen.

Example 11: The catheter of example 9, wherein the navigation wire is disposed radially outward of the outer catheter body.

Example 12: The catheter of any of examples 1-11, wherein the ultrasound transducer comprises a piezoelectric material.

Example 13: The catheter of example 12, wherein the piezoelectric material comprises a ceramic.

Example 14: The catheter of any of examples 1-13, wherein the transducer body is cylindrical.

Example 15: A catheter comprising: a first expandable member; a second expandable member disposed within the first expandable member; an outer catheter body defining an outer catheter lumen in fluid communication with the first expandable member; a fluid delivery tube disposed within the outer catheter lumen, the fluid delivery tube configured to deliver a first medium to the first expandable member; an inner tube disposed within the outer catheter lumen, the inner tube defining an inner tube lumen configured to deliver a second medium to the second expandable member to expand the second expandable member; and an ultrasound transducer disposed on an outer surface of the second expandable member.

Example 16: The catheter of example 15, wherein the second expandable member comprises an electrically conductive material.

Example 17: The catheter of example 16, wherein the electrically conductive material comprises a metallic foil.

Example 18: The catheter of example 16 or example 17, wherein the second expandable member is configured to electrically connect the ultrasound transducer to an external power source.

Example 19: The catheter of any of examples 15-18, further comprising a guidewire tube disposed within the inner tube lumen and extending through the first and second expandable members.

Example 20: The catheter of any of examples 15-18, further comprising a steerable tip at a distal end of the catheter.

Example 21: The catheter of example 20, further comprising a navigation wire configured to deflect the catheter.

Example 22: The catheter of example 21, wherein the navigation wire is disposed within the outer catheter lumen.

Example 23: The catheter of example 21, wherein the navigation wire is disposed radially outward of the outer catheter body.

Example 24: The catheter of any of examples 15-23, wherein the ultrasound transducer comprises a piezoelectric material.

Example 25: The catheter of example 24, wherein the piezoelectric material comprises a ceramic.

Example 26: A method comprising: navigating a catheter through vasculature of a patient to a target treatment site; expanding an expandable member of the catheter and circulating a fluid through the expandable member, wherein expanding the expandable member and circulating the fluid comprises: introducing the fluid into the expandable member through an inner tube lumen defined by an inner tube of the catheter, wherein the catheter further comprises an outer catheter body defining an outer catheter lumen in fluid communication with the expandable member, and wherein the inner tube is disposed within the outer catheter lumen; introducing the fluid into a transducer lumen of an ultrasound transducer of the catheter, the ultrasound transducer being positioned radially inward of the expandable member, wherein the ultrasound transducer comprises a transducer body comprising an inner surface defining the transducer lumen, and wherein at least a proximal portion of the transducer body is connected to a distal portion of the inner tube to fluidically connect the inner tube lumen and the transducer lumen, wherein the fluid flows from the transducer lumen into the expandable member through one or more fluid apertures of a transducer support lumen positioned at a distal portion of the transducer body, and from the expandable member to the outer catheter lumen; and delivering therapy to the target treatment site via a plurality of electrodes extending along at least a first portion of an outer surface of the transducer body and at least a second portion of the inner surface of the transducer body.

Example 27: The method of example 26, wherein the first portion of the outer surface and the second portion of the inner surface at least partially overlap in a radial direction.

Example 28: The method of example 26 or example 27, wherein the catheter further comprises a guidewire tube disposed within the inner tube lumen and the transducer lumen, the guidewire tube defining a guidewire lumen, and wherein navigating the catheter through the vasculature of the patient comprises navigating the catheter using a guidewire disposed within the guidewire lumen.

Example 29: The method of example 28, wherein the transducer support member electrically connects the plurality of electrodes to the guidewire tube.

Example 30: The method of any of examples 26-29, wherein the transducer support member is positioned between distal portion of the transducer body and the guidewire tube in a radial direction, and wherein the plurality of electrodes wrap around a proximal end of the transducer body.

Example 31: The method of example 30, wherein the plurality of electrodes define an active transducer area.

Example 32: The method of any of examples 26-31, wherein the catheter further comprises a steerable tip at a distal end of the catheter.

Example 33: The method of example 32, wherein navigating a catheter through vasculature of a patient to a target treatment site comprises deflecting a navigation wire disposed within the catheter.

Example 34: The method of example 33, wherein the navigation wire extends through the inner tube lumen and the transducer lumen.

Example 35: The method of example 33, wherein the navigation wire is disposed radially outward of the outer catheter body.

Example 36: The method of any of examples 26-35, wherein the ultrasound transducer comprises a piezoelectric material.

Example 37: The method of example 36, wherein the piezoelectric material comprises a ceramic.

Example 38: The catheter of any of examples 26-37, wherein the transducer body is cylindrical.

Example 39: A method comprising: navigating a catheter through vasculature of a patient to a target treatment site, the catheter comprising: a first expandable member; a second expandable member disposed within the first expandable member; an outer catheter body defining an outer catheter lumen in fluid communication with the first expandable member; a fluid delivery tube disposed within the outer catheter lumen, the fluid delivery tube configured to deliver a first fluid to the first expandable member; an inner tube disposed within the outer catheter lumen, the inner tube defining an inner tube lumen configured to deliver a second fluid to the second expandable member; and an ultrasound transducer disposed on an outer surface of the second expandable member; expanding the first expandable member of the catheter, wherein expanding the first expandable member comprises introducing a first medium into the first expandable member through the fluid delivery tube; expanding the second expandable member of the catheter, wherein expanding the second expandable member comprises introducing a second medium into the second expandable member through the inner tube; circulating the first medium through the first expandable member using the fluid delivery tube and the outer catheter lumen; and delivering a therapy to the target treatment site via the ultrasound transducer.

Example 40: The method of example 39, wherein the second expandable member comprises an electrically conductive material.

Example 41: The method of example 40, wherein the electrically conductive material comprises a metallic foil.

Example 42: The method of any of examples 40 and 41, wherein the second expandable member electrically connects the one or more ultrasound transducers to an external power source.

Example 43: The method of any of examples 39-42, wherein the catheter further comprises a guidewire tube disposed within the inner tube lumen, the guidewire tube defining a guidewire lumen, and wherein navigating the catheter through the vasculature of the patient comprises navigating the catheter using a guidewire disposed within the guidewire lumen.

Example 44: The method of any of examples 39-43, wherein the catheter further comprises a steerable tip at a distal end of the catheter.

Example 45: The method of any of examples 39-44, wherein navigating a catheter through vasculature of a patient to a target treatment site comprises deflecting a navigation wire disposed within the catheter.

Example 46: The method of example 45, wherein the navigation wire extends through the inner tube lumen.

Example 47: The method of example 45, wherein the navigation wire is disposed radially outward of the outer catheter body.

Example 48: The method of any of examples 39-47, wherein the one or more ultrasound transducers comprises a piezoelectric material.

Example 49: The method of example 48, wherein the piezoelectric material comprises a ceramic.

Further disclosed herein is the subject-matter of the following clauses:

1. A catheter comprising:
an expandable member;
an outer catheter body comprising an outer catheter lumen in fluid communication with the expandable member;
an inner tube positioned within the outer catheter lumen, the inner tube defining an inner tube lumen configured to deliver a fluid to the expandable member; and
an ultrasound transducer positioned in the expandable member, the transducer comprising:
   a transducer body comprising an inner surface defining a transducer lumen, wherein at least a proximal portion of the transducer body is connected to a distal portion of the inner tube to fluidically connect the inner tube lumen and the transducer lumen;
   a transducer support member positioned at a distal portion of the transducer body, the transducer support member defining one or more fluid apertures through which the fluid is configured to flow from the transducer lumen into the expandable member; and
   a plurality of electrodes extending along at least a first portion of an outer surface of the transducer body and at least a second portion of the inner surface of the transducer body.
   2. The catheter of clause 1, wherein the first portion of the outer surface and the second portion of the inner surface at least partially overlap in a radial direction.
   3. The catheter of clause 1 or clause 2, further comprising a guidewire tube disposed within the inner tube lumen and the transducer lumen, and extending through the expandable member, the guidewire tube defining a guidewire lumen.
   4. The catheter of clause 3, wherein the transducer support member electrically connects the plurality of electrodes to the guidewire tube.
   5. The catheter of clause 3 or clause 4, wherein the transducer support member is positioned between distal portion of the transducer body and the guidewire tube in a radial direction, and wherein an electrode of the plurality of electrodes wraps around a proximal end of the transducer body.
   6. The catheter of any one of clauses 1-5, wherein the plurality of electrodes define an active transducer area.
   7. The catheter of clause 1, further comprising a steerable tip at a distal end of the catheter, wherein the catheter does not include a guidewire lumen.
   8. The catheter of clause 7, further comprising a navigation wire configured to cause the catheter to deflect, wherein the navigation wire extends through the inner tube lumen and the transducer lumen or wherein the navigation wire is disposed radially outward of the outer catheter body.
   9. The catheter of any one of clauses 1-8, wherein the ultrasound transducer comprises a piezoelectric material, and, optionally, wherein the piezoelectric material comprises a ceramic.
   10. A catheter comprising:
a first expandable member;
a second expandable member disposed within the first expandable member;
an outer catheter body defining an outer catheter lumen in fluid communication with the first expandable member;
a fluid delivery tube disposed within the outer catheter lumen, the fluid delivery tube configured to deliver a first medium to the first expandable member;
an inner tube disposed within the outer catheter lumen, the inner tube defining an inner tube lumen configured to deliver a second medium to the second expandable member to expand the second expandable member; and
an ultrasound transducer disposed on an outer surface of the second expandable member.
   11. The catheter of clause 10, wherein the second expandable member comprises an electrically conductive material.
   12. The catheter of clause 11, wherein the electrically conductive material comprises a metallic foil.
   13. The catheter of clause 11 or clause 12, wherein the second expandable member is configured to electrically connect the ultrasound transducer to an external power source.
   14. The catheter of any of clauses 10-13, further comprising a guidewire tube disposed within the inner tube lumen and extending through the first and second expandable members.
   15. The catheter of any of clauses 10-13, further comprising a steerable tip at a distal end of the catheter.
   16. The catheter of clause 15, further comprising a navigation wire configured to deflect the catheter, wherein the navigation wire extends through the inner tube lumen and the transducer lumen or wherein the navigation wire is disposed radially outward of the outer catheter body.
   17. The catheter of any of clauses 10-16, wherein the ultrasound transducer comprises a piezoelectric material, and, optionally, wherein the piezoelectric material comprises a ceramic.

1. A catheter comprising:
an expandable member;
an outer catheter body comprising an outer catheter lumen in fluid communication with the expandable member;
an inner tube positioned within the outer catheter lumen, the inner tube defining an inner tube lumen configured to deliver a fluid to the expandable member; and
an ultrasound transducer positioned in the expandable member, the transducer comprising:
a transducer body comprising an inner surface defining a transducer lumen, wherein at least a proximal portion of the transducer body is connected to a distal portion of the inner tube to fluidically connect the inner tube lumen and the transducer lumen;
a transducer support member positioned at a distal portion of the transducer body, the transducer support member defining one or more fluid apertures through which the fluid is configured to flow from the transducer lumen into the expandable member; and
a plurality of electrodes extending along at least a first portion of an outer surface of the transducer body and at least a second portion of the inner surface of the transducer body.

2. The catheter of claim 1, wherein the first portion of the outer surface and the second portion of the inner surface at least partially overlap in a radial direction.

3. The catheter of claim 1, further comprising a guidewire tube disposed within the inner tube lumen and the transducer lumen, and extending through the expandable member, the guidewire tube defining a guidewire lumen.

4. The catheter of claim 3, wherein the transducer support member electrically connects the plurality of electrodes to the guidewire tube.

5. The catheter of claim 3, wherein the transducer support member is positioned between distal portion of the transducer body and the guidewire tube in a radial direction, and wherein an electrode of the plurality of electrodes wraps around a proximal end of the transducer body.

6. The catheter of claim 1, wherein the plurality of electrodes define an active transducer area.

7-8. (canceled)

9. The catheter of claim 1, wherein the ultrasound transducer comprises a piezoelectric material.

10. A catheter comprising:
a first expandable member;
a second expandable member disposed within the first expandable member;
an outer catheter body defining an outer catheter lumen in fluid communication with the first expandable member;
a fluid delivery tube disposed within the outer catheter lumen, the fluid delivery tube configured to deliver a first medium to the first expandable member;
an inner tube disposed within the outer catheter lumen, the inner tube defining an inner tube lumen configured to deliver a second medium to the second expandable member to expand the second expandable member; and
an ultrasound transducer disposed on an outer surface of the second expandable member.

11. The catheter of claim 10, wherein the second expandable member comprises an electrically conductive material.

12. The catheter of claim 11, wherein the electrically conductive material comprises a metallic foil.

13. The catheter of claim 11, wherein the second expandable member is configured to electrically connect the ultrasound transducer to an external power source.

14. The catheter of claim 10, further comprising a guidewire tube disposed within the inner tube lumen and extending through the first and second expandable members.

15-16. (canceled)

17. The catheter of claim 10, wherein the ultrasound transducer comprises a piezoelectric material, and, optionally, wherein the piezoelectric material comprises a ceramic.

18. The catheter of claim 9, wherein the piezoelectric material comprises a ceramic.

19. The catheter of claim 1, wherein the catheter does not include a guidewire lumen, wherein the catheter further comprises:
a steerable tip at a distal end of the catheter; and
a navigation wire configured to cause the catheter to deflect, wherein the navigation wire extends through the inner tube lumen and the transducer lumen.

20. A method comprising:
navigating a catheter through vasculature of a patient to a target treatment site;
expanding an expandable member of the catheter and circulating a fluid through the expandable member, wherein expanding the expandable member and circulating the fluid comprises:
introducing the fluid into the expandable member through an inner tube lumen defined by an inner tube of the catheter, wherein the catheter further comprises an outer catheter body defining an outer catheter lumen in fluid communication with the expandable member, and wherein the inner tube is disposed within the outer catheter lumen;
introducing the fluid into a transducer lumen of an ultrasound transducer of the catheter, the ultrasound transducer being positioned radially inward of the expandable member, wherein the ultrasound transducer comprises a transducer body comprising an inner surface defining the transducer lumen, and wherein at least a proximal portion of the transducer body is connected to a distal portion of the inner tube to fluidically connect the inner tube lumen and the transducer lumen,
wherein the fluid flows from the transducer lumen into the expandable member through one or more fluid apertures of a transducer support lumen positioned at a distal portion of the transducer body, and from the expandable member to the outer catheter lumen; and
delivering therapy to the target treatment site via a plurality of electrodes extending along at least a first portion of an outer surface of the transducer body and at least a second portion of the inner surface of the transducer body.

21. The method of claim 20, wherein the first portion of the outer surface and the second portion of the inner surface at least partially overlap in a radial direction.

22. The method of claim 20, wherein the catheter further comprises a guidewire tube disposed within the inner tube lumen and the transducer lumen, the guidewire tube defining a guidewire lumen, and wherein navigating the catheter through the vasculature of the patient comprises navigating the catheter using a guidewire disposed within the guidewire lumen.

23. The method of claim 22, wherein the transducer support member is positioned between distal portion of the transducer body and the guidewire tube in a radial direction, and wherein the plurality of electrodes wrap around a proximal end of the transducer body.

24. The method of claim 20, wherein the ultrasound transducer comprises a piezoelectric material.
